(19) United States
(12) Patent Application Publication
(10) Pub. No.: US 2016/0230167 A1
(43) Pub. Date: Aug. 11, 2016

(54) Method and Apparatus for Reprogramming Living Cells

(71) Applicant: JenLab GmbH, Saarbruecken (DE)

(72) Inventors: Karsten KOENIG, Saarbruecken (DE); Aisada UCHUGONOVA, Saarbruecken (DE)

(21) Appl. No.: 15/019,053

(22) Filed: Feb. 9, 2016

(30) Foreign Application Priority Data
Feb. 9, 2015 (DE) 10 2015 101 838.1

(51) Int. Cl. C12N 13/00; C12N 5/074

(52) U.S. Cl. CPC C12N 13/00; C12N 5/0696; C12N 2529/00; C12N 2501/65; C12N 2501/60

(57) ABSTRACT

A method and an apparatus for reprogramming living cells without using viruses. In that method a cocktail comprising at least two transcription factors and a microRNA is transfected into the interior of at least one cell in order to convert this cell into iPS cells or into another type of cell, by storing the cells to be converted in an aqueous environment of the cocktail without viral carriers and focusing a femtosecond laser in a laser scanning microscope with a numerical aperture between 0.9 and 1.5 on a cell membrane of the cell to be reprogrammed and controlling the position of the focus. The exposure period and laser power for the optical treatment of the cell such that the focus depending on the pulse repetition frequency with an output between 7 mW and 100 mW generates a transient small-pore hole with a size up to 500 nm.

### RELATED APPLICATIONS

This application claims priority to German Patent Application No. DE 10 2015 101 838.1, filed Feb. 9, 2015 which is incorporated herein by reference in its entirety.

### FIELD OF THE INVENTION

The invention is directed to a method and an apparatus for reprogramming living cells, particularly for virus-free reprogramming of adult and embryonic stem cells, iPS cells and already differentiated cells.

### BACKGROUND OF THE INVENTION

Medical use of human adult and embryonic stem cells entails a number of problems. Adult stem cells are difficult to isolate, often only possess the possibility of differentiating into specific tissue cells (multipotent cells) and are often damaged when extracted from patients.

Embryonic human stem cells, however, are pluripotent. They can differentiate into any cell type and can renew themselves while retaining pluripotency. However, obtaining these cells requires the use of fertilized human ova. The possibilities for clinical use are limited on bioethical grounds. Further, immunosuppressive agents must be administered because of the risk of rejection in regenerative medicine when using a graft produced from human embryonic stem cells.

The above-mentioned drawbacks in the clinical use of stem cells do not arise when using induced pluripotent stem cells (iPS). These iPS cells are generated by reprogramming already differentiated cells to form undifferentiated cells (reversion) which behave like the embryonic stem cells. The exact mechanism, however, is unknown. Human iPS cells were produced from human fibroblasts for the first time in 2007 (Takahashi et al., Cell 131 (2007) 861-872).

There are various methods of reprogramming However, many methods such as cell nucleus transfer or cell transfusion again require the use of ova.

Direct programming, in which typically three or four specific transcription factors or microRNA are transfected into differentiated cells, does not have this disadvantage. It has been assumed heretofore that the transcription factors must remain stable for at least one week. For this reason, they have been integrated and applied in genomic DNA of a retrovirus or lentivirus (Miyazaki et al., Jpn. J. Clin. Oncol. 42 (2012) 773-779). These pioneers of viral reprogramming of cells were awarded the Nobel prize for medicine and physiology in 2012.

Aside from the possibility of first producing iPS cells through reprogramming and subsequently selectively differentiating them for medical use, there is also the possibility of partial reprogramming In this case, no pluripotent iPS cells are generated, but rather either the required differentiated cells are generated directly, e.g., a heart cell from a skin cell, or multipotent cells are generated which can only differentiate into specific cells (Efe et al., Nature Cell Biology 13 (2011) 215-222; Vierbuchen et al., Nature 463 (2010) 7284). Viruses are also commonly used in this case, but three transcription factors are often sufficient. With partial reprogramming, the hope is also to avoid uncontrolled effects such as the generation of cancerous tissue and unpredictable cell mutations.

To date, clinical use of human reprogrammed cells has been limited or rendered impossible by the use of viruses. There is the further disadvantage of low conversion efficiency, i.e., too few vital cells in proportion to lethal cells after viral injection transcription.

A further disadvantage consists in that the method for producing iPS cells in the form of three-dimensional clusters (iPS colony) is cumbersome and currently takes at least one week.

Required grafts are produced from the iPS cells or partially reprogrammed cells in complicated steps under in vitro conditions. In addition, iPS cells could be deposited directly into a target tissue by injection. To date, only embryonic and adult stem cells have been injected directly into the damaged target tissue (e.g., into the heart muscle, spinal cord). It is hoped in this way to obtain efficient conversions into the required differentiated cell type, since the environment is already made up of these cells and suitable differentiation factors are available. A reprogramming of cells within tissue is currently impossible. Further, in-situ reprogramming within the tissue to be treated in the patient would be of especially great interest.

Apart from its application in regenerative medicine, reprogramming of cancer stem cells in the patient's body in particular would be of great interest.

Accordingly, the following problem areas exist:

a) the use of viruses limits clinical use,

b) the efficiency of obtaining reprogrammed cells is low,

c) the production of reprogrammed cells is generally cumbersome,

d) the production of reprogrammed cells in a tissue environment is impossible,

e) the reprogramming of cells in diseased target tissue in a patient by viral methods is impossible.

Various methods are currently being researched which would allow direct reprogramming without the use of viruses. Accordingly, a reprogramming of mouse skin cells was implemented by the so-called STAP method (stimulus-triggered acquisition of pluripotency) by a Japanese research team based on variation of the microenvironment as published in the prestigious journal Nature (Obokata et al., Nature 505 (2014) 641-647). However, the method has not yet been corroborated by other research groups. To date, it has only been applied in animal cells. Moreover, a large number of cells die because the microenvironment has been modified to transient extreme pH gradients. A reprogramming of cells in a tissue environment is not provided with this method.

Heretofore, the reprogramming of cells has usually been carried out using viruses which hinder clinical application. To date, the new STAP method is limited to animal cells and entails a high mortality rate. Additionally, all reprogramming has so far been carried out with a low efficiency and cumbersome, time-consuming preparatory steps. To date, direct reprogramming in an in vitro tissue or directly in vivo in the patient has been impossible.

### SUMMARY OF THE INVENTION

Therefore, it is the object of the invention to find a novel possibility for direct, efficient and fast cell reprogramming without using viruses and with the option of direct reprogramming in vital tissue.

According to the invention, this object is met by a method for reprogramming living cells in which a cocktail comprising at least two transcription factors and a microRNA is transfected into the interior of at least one cell in order to convert this cell into iPS cells or into another type of cell, having the following steps:

preparation of the cocktail without viral carriers in an aqueous environment of the at least one cell to be reprogrammed,

providing a radiation source in the form of a femtosecond laser with a pulse repetition frequency ranging between 50 MHz and 2 GHz with a wavelength in the range of from 700 to 1200 nm,

focusing a laser beam of the femtosecond laser by means of a laser scanning microscope with a numerical aperture of between 0.9 and 1.5 on a cell membrane of a selected cell in a sample with the at least one cell on a displaceable x-y table,

directing an attenuated, nondestructive laser beam of the femtosecond laser, which is also used for optical treatment, to the device and observing the at least one selected cell in the focus of the laser scanning microscope by means of a scanner, and

controlling the position of the at least one cell in the focus, the exposure period and laser power for the optical treatment such that the focus depending on the pulse repetition frequency with an output between 7 mW and 100 mW generates a transient small-pore hole with a size in the range of up to 500 nm within the cell membrane of the cell in order to allow a diffusion of the cocktail for multiple reprogramming of the cell through the cell membrane into the interior of the cell such that a virus-free optical multiple reprogramming takes place.

The irradiation for reprogramming by means of a femtosecond laser is preferably carried out at a frequency between 75 and 85 MHz and with a center wavelength between 700 and 900 nm by means of the laser scanning microscope via a microscope objective with a high numerical aperture between 1.1 and 1.3.

In a preferred embodiment, the irradiation for reprogramming is carried out at an output of between 7 and 20 mW with pulse lengths between 5 fs and 20 fs and for a duration of between 0.2 and 1 second.

It is particularly advisable to carry out the irradiation for reprogramming at an output of between 50 and 100 mW with pulse lengths between 100 fs and 200 fs and for a duration of between 0.2 and 1 second.

Cells to be reprogrammed are preferably provided as monolayer cells on a glass substrate on the x-y table and are covered with the virus-free cocktail in aqueous solution before irradiating for reprogramming

In an alternative variant of the method, cells to be reprogrammed are streamed as aqueous cell suspension with the virus-free cocktail into a microfluidic flow cell through a micro-cannula. The irradiation for reprogramming is preferably carried out with an output between 50 mW and 100 mW using a shaped laser beam in Bessel beam mode with elongated focus, the laser beam forms an elongated focus over the entire diameter of the micro-cannula, and the scanner of the laser scanning microscope carries out a line scan orthogonal thereto so as to cover an entire cross-sectional area of the micro-cannula, and the cell suspension flows through the micro-cannula with a flow rate of 135 to 145 nl/s.

In this connection, it is advisable that a flow is circulated through the flow cell so that the cell suspension of cells to be reprogrammed and virus-free cocktail can stream through the micro-cannula repeatedly (up to three repetitions) in order to increase the hit ratio of cells to be reprogrammed with the scanned elongated focus.

After a diffusion time of at least five seconds after irradiation, the plasmid cocktail around the cells to be reprogrammed is advantageously replaced by plasmid-free medium, and the cells are incubated and stored in the plasmid-free medium in an incubator for at least two days.

It has proven advisable to monitor the results of the optical reprogramming during storage in the incubator by means of a fluorescence microscope through evidence of a green fluorescent protein which has been co-transfected during reprogramming through the transient hole in the cell membrane.

An optical multiple reprogramming of the at least one cell to an iPS cell is advantageously carried out by means of optical treatment.

In a further advantageous embodiment, a direct optical reprogramming can be carried out by means of the optical treatment of the at least one cell through conversion of one cell type into another cell type.

Further, the optical treatment can be utilized in a tissue formed of a three-dimensional cell complex as perforation by boring channels with a diameter of up to 10 μm.

The above-stated object is further met in an apparatus for reprogramming living cells in which a cocktail made up of a required microRNA and at least two plasmids as transcription factors for the reprogramming is transfected into the interior of at least one cell to be reprogrammed in order to convert it into iPS cells or into another type of cell, which apparatus is characterized in that a femtosecond laser is provided for irradiation at a frequency between 75 and 85 MHz and a center wavelength between 750 and 900 nm to generate a virus-free optical reprogramming through selective perforation of a cell membrane for transfecting the cocktail into the interior of the at least one cell to be reprogrammed, and in that there is provided a laser scanning microscope which is outfitted with the femtosecond laser and which has a microscope objective with a high numerical aperture between 0.9 and 1.5, with respect to which laser scanning microscope the cells to be reprogrammed can be arranged such that cells to be reprogrammed can be continuously selected for irradiation in order to achieve a perforation with at least one transient small-pore hole having a size in the range of up to 500 nm within the cell membrane of the at least one cell to be reprogrammed.

An x-y table is preferably provided by which the positioning of monolayer cells on a glass substrate can be carried out for focusing a laser beam on the cell membrane of the monolayer cells.

The femtosecond laser is advisably configured as a femtosecond laser with a frequency between 75 and 85 MHz, a pulse length between 10 fs and 20 fs and a center wavelength between 750 and 900 nm and which can be focused on the cell membrane by means of the microscope objective having a high numerical aperture in the range of from 1.1 to 1.3 with a focus in the submicrometer range of up to 500 nm.

In a particularly advantageous manner, the x-y table has a microfluidic flow cell with a micro-cannula through which flows an aqueous cell suspension of the cocktail with the cells to be reprogrammed.

The femtosecond laser is preferably configured to emit a Bessel beam with an elongated focus, and the diameter of the micro-cannula is fully covered by an elongated focus of the Bessel beam that is moved in a line scan by means of a scanner of the laser scanning microscope such that the cells to be reprogrammed pass through the focus at a flow velocity generated by a flow rate between 135 and 145 nl/s with a cannula diameter of 100 μm and are impinged upon while flowing through.

A cell chamber is advisably arranged downstream of the flow cell to capture the cell suspension of reprogrammed cells and cocktail and for replacing the cocktail with a plasmid-free medium for storage in an incubator.

The invention is based on the fundamental idea that the efficiency of cell reprogramming can be decisively increased through the use of optical reprogramming based on ultrashort laser pulses for transfecting the required DNA plasmids and microRNA. In this respect, a transient alteration of the permeability of the cell membrane is brought about in that the latter is perforated by fs laser pulses and transcription factors for reprogramming are transferred into the interior of the cell.

Ultrashort laser pulses are used on principle for the optical cell reprogramming according to the invention as is known for laser-based permanent transfection of DNA into living cells (U.S. Pat. No. 7,892,837 B2), wherein the laser causes transient membrane holes to be generated. A flow cytometer for femtosecond laser perforation of cells has also been described for this purpose in WO 2013/120960 A1. Heretofore, however, only one gene has been transfected by laser action, generally a plasmid which produces a green fluorescence (GFP protein). However, reprogramming requires transfection of multiple genes or reprogramming factors (usually four for producing iPS cells). On the other hand, the use of lasers instead of problematic viral reprogramming of cells is a completely novel approach which, as “sterile optical reprogramming”, permits low-risk reprogramming of cells also in three-dimensional cell complexes and potentially within the human body.

Preliminary work has shown that an optical, direct, virus-free reprogramming, particularly for producing human iPS cells from human fibroblasts, is possible using femtosecond lasers. For this purpose, the human cells were added to a medium containing four transcription factors, Oct-4, NANOG, Lin-28 Sox2 and a GFP plasmid (green fluorescent protein as marker), and were irradiated with twelve femtosecond pulses of a 85-MHz titanium : sapphire laser for a few milliseconds (50-100 ms). Astonishingly, a single bombardment of the cells in a special flow cytometer is sometimes sufficient to generate a plurality of iPS colonies which exhibit green fluorescence as a result of the additional transfection of the GFP plasmid. Interestingly, iPS cell clusters (embryoic bodies) were already generated after 3 to 5 days and, therefore, considerably faster compared to viral direct reprogramming Aside from the achieved rapidity, the high efficiency of the optical reprogramming compared to viral methods is surprising. Accordingly, iPS cells can be produced on the one hand or a direct conversion of one cell type into another cell type (i.e., so-called direct reprogramming) can take place, which has only been partially successful in the art to date (see, e.g., Efe et al., Vierbuchen et al., both cited above, Szabo et al.: Direct conversion of human fibroblasts to multilineage blood progenitors, Nature 485 [2012] 585, or Kim: Converting human skin cells to neurons: a new tool to study and treat brain disorders?, Cell Stem Cell 9 [2011] 179).

In principle, the laser system for optical reprogramming can also be employed directly in three-dimensional collections of cells. To this end, transcription factors must be introduced into the microenvironment. This can be carried out, e.g., through mechanical injection or, in accordance with the invention, through optical generation of defined microchannels from the surface of the tissue to the target site in the tissue by means of a femtosecond laser system. The femtosecond laser system shall then also be utilized to produce the required membrane pores for transfecting the transcription factors from the microenvironment into the cell. Accordingly, target tissue can be reprogrammed in a spatially selective manner This is usually carried out outside the human body, e.g., within the framework of tissue engineering. In principle, however, the method according to the invention can also be utilized within the body of the patient.

Heretofore, medically compatible femtosecond laser systems such as multiphoton tomographic apparatus for skin analysis and systems for the treatment of vision defects have not been suitable for enabling optical reprogramming in patient tissue. In tomography equipment, typical 80-MHz laser pulses with a length of 100 fs to 200 fs and an average output of typically 15 mW with conventional radiation dwell times of less than 100 μs are only suitable for generating exclusively visual imaging of the tissue through two-photon fluorescence or frequency doubling (second harmonic generation—SHG). By contrast, femtosecond lasers for the treatment of defective vision rely on destructive, photodisruptive effects by means of high-energy femtosecond laser pulses in the kHz range by which plasma-filled bubbles and shockwaves are generated which destroy tissue structures, including the stable collagen network and whole cells. Selective action upon individual cells in order to bore a transient channel into the membrane of the cell without causing irreversible damage to the cell is definitely impossible.

The optical, virus-free, complete or partial reprogramming of cells in accordance with the invention opens up entirely novel therapeutic possibilities, particularly in the field of regenerative medicine (e.g., the production of graft tissues) and in the treatment of cancers.

### BRIEF DESCRIPTION OF THE DRAWINGS

The invention will be described more fully in the following with reference to embodiment examples and drawings. The drawings show:

FIG. 1 is a schematic view of an arrangement according to the invention for implementing the method;

FIG. 2 is a schematic view of a first embodiment form of an apparatus according to the invention for treating a monolayer cell complex;

FIG. 3 is a schematic view of a second embodiment form of an apparatus according to the invention with a flow cell device, including a possible multiple treatment of the cells flowing through;

FIG. 4 is an enlarged detail of the flow cell according to FIG. 3 showing a scanned laser beam in the form of a Bessel beam with elongated focus.

### DETAILED DESCRIPTION OF THE PREFERRED EMBODIMENTS

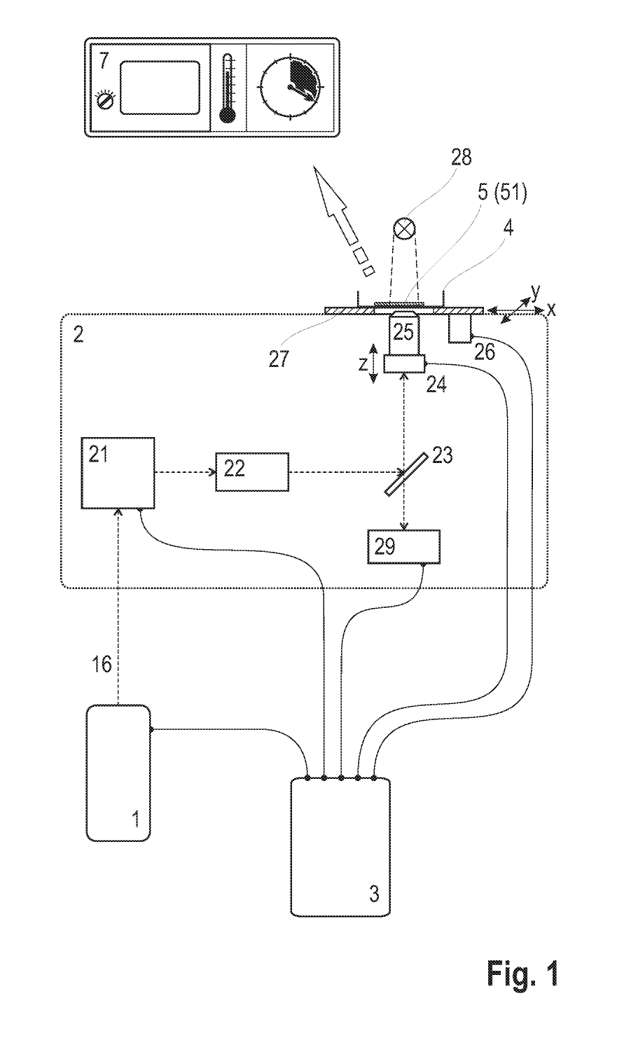

FIG. 1 shows the basic construction of an apparatus according to the invention comprising a radiation source 1, a laser scanning microscope 2 and a control unit 3. The laser scanning microscope 2 has a x-y table 27 on which a sample 5 is held and which can be moved in a horizontal x and y direction. In order to observe the sample 5 through a microscope objective 25, the laser scanning microscope 2 has illumination 28 and a video camera 29. The movement of the x-y table 27 is carried out by means of an x-y table drive 26. The movement of the microscope objective 25 for focusing in a vertical z direction is carried out by means of a focusing drive 24. The x-y table drive 26 and focusing drive 24 are connected to the control unit 3.

The radiation source 1 delivers a pulsed laser beam 16 with pulse lengths in the focus of the laser scanning microscope 2 in the range of 5 to 250 fs, preferably in the range of between 5 and 200 fs, particularly preferably between 5 and 30 fs, and a high pulse repetition frequency in the range of from 50 MHz to 2 GHz, preferably in the range of between 70 and 1000 MHz. In a particularly preferred variant, the laser beam 16 has pulse lengths of 10 to 20 fs and a pulse repetition frequency of between 75 and 85 MHz.

Cell membranes of cells in the sample 5 are transiently perforated by the laser beam 16. To this end, the laser beam 16 is deflected along the beam path by means of a scanner 21 and, after passing through a beam expansion 22, is deflected by a beamsplitter 23 in the direction of the sample 5, where it is focused in the sample 5 through the microscope objective 25 with a very high numerical aperture between 0.9 and 1.5, preferably between 1.1 and 1.3. The scanner 21 moves the focused laser beam 16 over a determined region of the sample 5 such that the quantity of cells impinged by the focused laser beam 16 can be increased. The beamsplitter 23 allows the sample 5 to be observed while the laser beam 16 is used. The timing and power of the laser beam 16 are likewise controlled by the control unit 3.

The laser beam 16 is controlled by a shutter 12 for temporally limiting the laser action on the cell membrane and for boring channels in a cell complex (hereinafter, tissue) and by a control unit 3 for power switching between a nondestructive radiation mode of the laser beam 16 for adjustment and observation of the cells and a perforation mode of the laser beam 16 for generating transient small-pore holes in the cell membrane (pore size 10 nm to 500 nm) for purposes of transferring (temporary diffusion) microRNA and transcription factors (in the form of plasmids, episomal vectors, transposons).

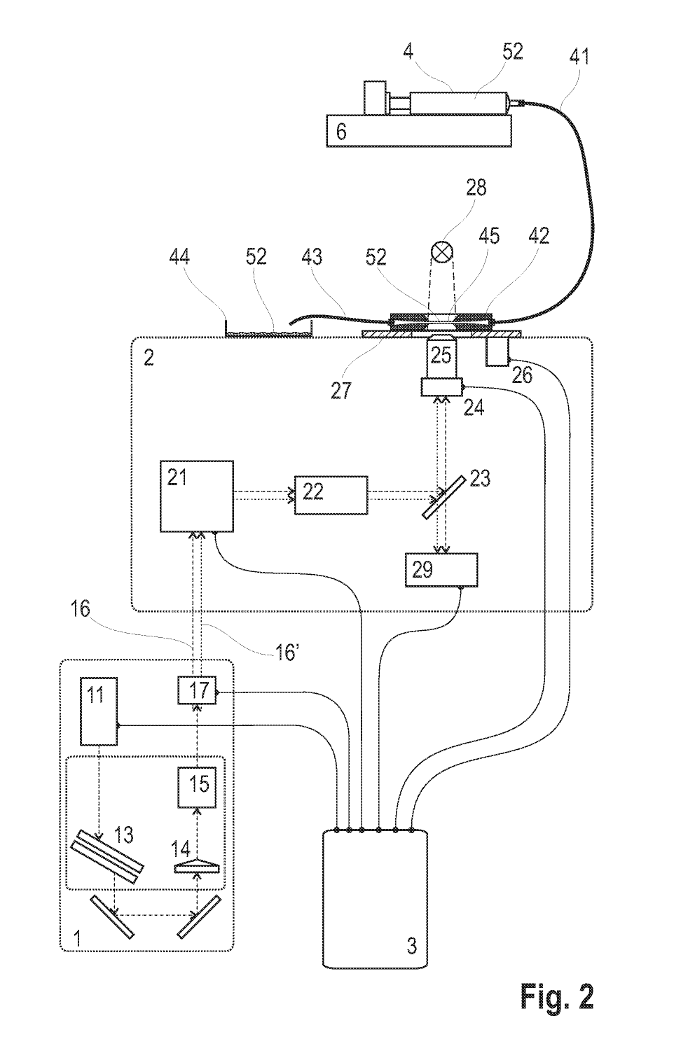

In order to achieve small-pore holes of this kind in the cell membrane, the laser beam 16 is influenced with respect to the light distribution thereof before entering the laser scanning microscope 2 by a beam-shaping unit which can include a dispersion compensator 13, preferably in the form of chirped mirrors, an Axikon element 14 for generating a coaxial illumination ring, and periscope optics 15 as shown by way of example in FIG. 2. Further, the laser beam 16 is influenced by an attenuation unit 17 for controlling the output power of the laser. All of these units improve the homogeneity and radial intensity distribution toward an increase in the edge intensity of the initial Gaussian bundle for sharper focusing of the laser beam 16 in the target volume of the sample 5 and its output power for the observation mode and for the perforation mode.

### EMBODIMENT EXAMPLE 1

In a first embodiment example, human skin cells marketed by LONZA (#CC-2511) are cultured as monolayer cells in a supply receptacle 4. For the reprogramming of cells, a solution covering the monolayer cells 51 is added to the supply receptacle 4. The solution contains a plasmid mixture marketed by SBI (System Biosciences pMC-LGNSO MiniCircle DNA, #SRM100A-1) with plasmids Oct-4, Lin-28, NANOG, Sox2+GFP in a concentration of 5-10 μg/ml.

Human skin cells marketed by LONZA (#CC-2511) were cultured as monolayer cells 51 in a supply receptacle 4 with a glass bottom having a thickness of 160 μm which produces a working distance of 170 μm from the cells to be treated. A suspension in the form of a plasmid cocktail marketed by SBI (System Biosciences, pMC-LGNSO MiniCircle DNA, #SRM100A-1) containing plasmids Oct-4, Lin-28, NANOG, Sox2+GFP in a concentration of 5-10 μg/ml is added as solution to the monolayer cells 51 for biochemical implementation of the reprogramming step. The thickness of the glass bottom of the supply receptacle 4 corresponds to approximately 40 times the numerical aperture of the microscope objective 25 which, in this case, has a numerical aperture of 1.3.

The cells which have been prepared in this way are then exposed by means of a laser scanning microscope 2 to irradiation by a femtosecond laser 11 (10 fs, 85 MHz, center wavelength 800 nm) by means of a microscope objective 25 with a high numerical aperture of 1.3.

In principle, the femtosecond laser 11 can preferably have a pulse repetition frequency of between 80 and 85 MHz and pulse lengths of between 10 and 250 fs and can be used with wavelengths ranging from 700 to 1200 nm When a pulse length of 100 to 200 fs is used in the focus of the laser beam 16, a mean power of 50 to 100 mW must be set; however, with pulse lengths between 10 and 20 fs a mean power of only 7 to 15 mW is adjusted so that the individual cells are not destroyed during the perforation of the cell membrane.

To find a suitable membrane position in a selected cell, a motor-driven x-y table 27 is moved in such a way that the focus of the attenuated, nondestructive laser beam 16 is positioned on the cell membrane. The attenuated, nondestructive laser beam 16′ which is required solely for imaging by means of video camera 29 is operated at an output below 5 mW.

The mean output of the laser beam 16 is then increased to approximately 10-15 mW and the membrane is irradiated for 50-100 ms. In this way, it is also possible to perforate an individual cell at up to three positions for each individual exposure.

The destructive effect of boring a transient hole with a diameter in the range of from 10 to 500 nm is achieved through the formation of a plasma-filled cavitation bubble. It is brought about by means of a flash vaporization of the volume of the cell membrane located in the focus and is recorded by the video camera 29. The cavitation bubble which has a maximum size of 5 μm disappeared in some experiments after approximately 5 seconds. Within this time, the plasmid cocktail was able to diffuse into the cell.

After irradiation, the cocktail medium is exchanged for a plasmid-free medium and the cells are stored in the incubator 7 under a gas atmosphere of 5% CO₂ and 95% air at 37° C. Proof that the plasmids have been taken into the cell DNA can be furnished by the formation of the added green fluorescent GFP protein using a fluorescence microscope. The green fluorescence usually occurs within 12 to 36 seconds after laser irradiation. Three-dimensional green fluorescent cell clusters arise over the course of the next five days with a morphology corresponding to that of virus-generated cell clusters (embryoic bodies).

### EMBODIMENT EXAMPLE 2

Human skin cells marketed by LONZA (#CC-2511) in a cell suspension 52 containing a plasmid cocktail marketed by SBI (System Biosciences), pMC-LGNSO MiniCircle DNA, #SRM100A-1 with plasmids Oct-4, Lin-28, NANOG, Sox2+GFP (in a concentration that is three to four times higher than that applied to the monolayer cells 51 in Example 1) are added to a receptacle of a metering device 6.

In an apparatus shown in FIG. 2, the cell suspension 52 is supplied to a flow cell 42 through line 41 from the metering device 6 which can be a conventional syringe with a linear plunger feed. The flow cell 42 comprises a micro-cannula 45 which in this example has an inner diameter of 100 μm and in which the skin cells are virtually isolated, if permitted by the cell suspension 52, to flow through the micro-cannula 45 at a typical flow velocity of 18 μm/ms and a flow rate of 139 nl/s.

After passing through the microscope optics 25 of a laser scanning microscope 2, a laser beam 16 of the femtosecond laser 11 having a beam profile shaped into a quasi-Bessel beam by the beam-shaping unit comprising dispersion compensator 13, Axikon element 14 and periscope optics 15 has an elongated focus over the entire diameter of the micro-cannula 45 and impinges with a repetition frequency of 80 MHz such that it is constantly perpendicular to the direction of the micro-cannula 45 and, in so doing, is moved by the scanner 21 orthogonal thereto in a line scan at 7 to 30 ms per line so as to permeate an inner cross-sectional area of the micro-cannula 45 in a continuous, practically two-dimensional manner and accordingly perforates a majority of the cells to be reprogrammed (e.g., human skin cells) inside micro-cannula 45 of flow cell 42. The maximum mean output of the laser beam 16 is 135 mW in quasi-Bessel beam mode (utilizing a 10×, 1.13 NA objective for focusing the laser pulses over the entire inner cross-sectional area of the micro-cannula 45).

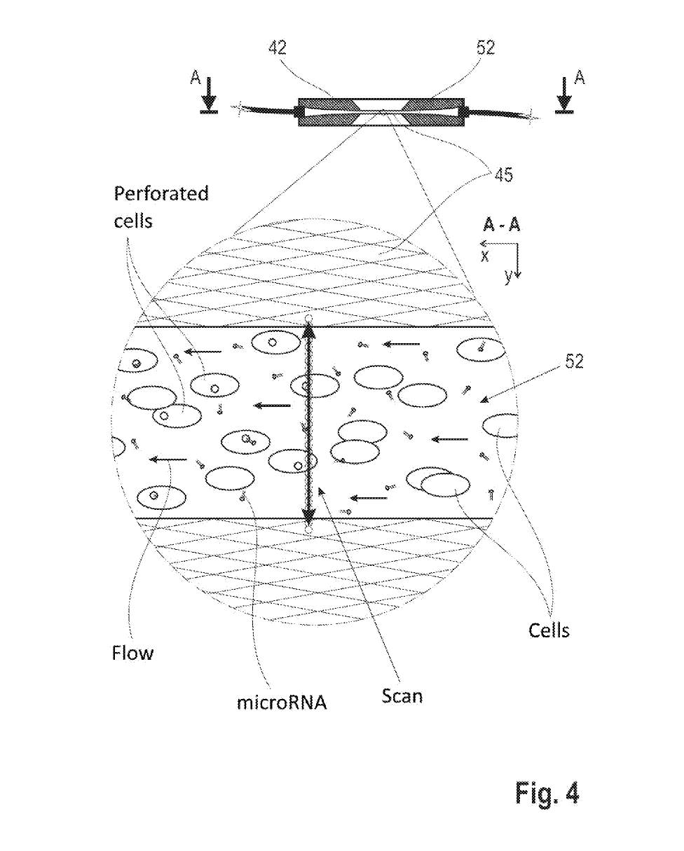

To illustrate the continuous permeation of the micro-cannula 45 by the scanned elongated laser focus as the cell suspension 52 is streamed through, FIG. 4 shows a schematic view of a section of the micro-cannula 45 in plane A-A. The microscope objective 25 (not shown) is directed from above onto plane A-A in the drawing. The elongated focus extends perpendicular to the drawing plane and covers the inner diameter of the micro-cannula 45 in depth direction (downward in z direction). The y direction is swept by the scanner 21 of the laser scanning microscope 2 vertical to the drawing plane and leads to a permanent quasi-two-dimensional formation of the focus over the entire inner cross-sectional area of the micro-cannula 45. In this way, with streaming cell suspension 52, a high efficiency of the optical reprogramming is already achieved through progressive perforation of the cells in the stream of cell suspension 52.

All the rest of the parameters and processes of the laser irradiation are carried out in the same manner as in Example 1.

The cells are subsequently removed from the micro-cannula 45 to a standardized cell chamber 44 via an outlet 43 and are captured therein and then washed in a growth medium and incubated and stored with the growth medium in the incubator 7 at 37° C. under 5% CO₂ and 96% air. The cells which exhibit green fluorescence as a result of successful transfection are then detected by a fluorescence microscope and separated by centrifuging after the usual period of two to five days.

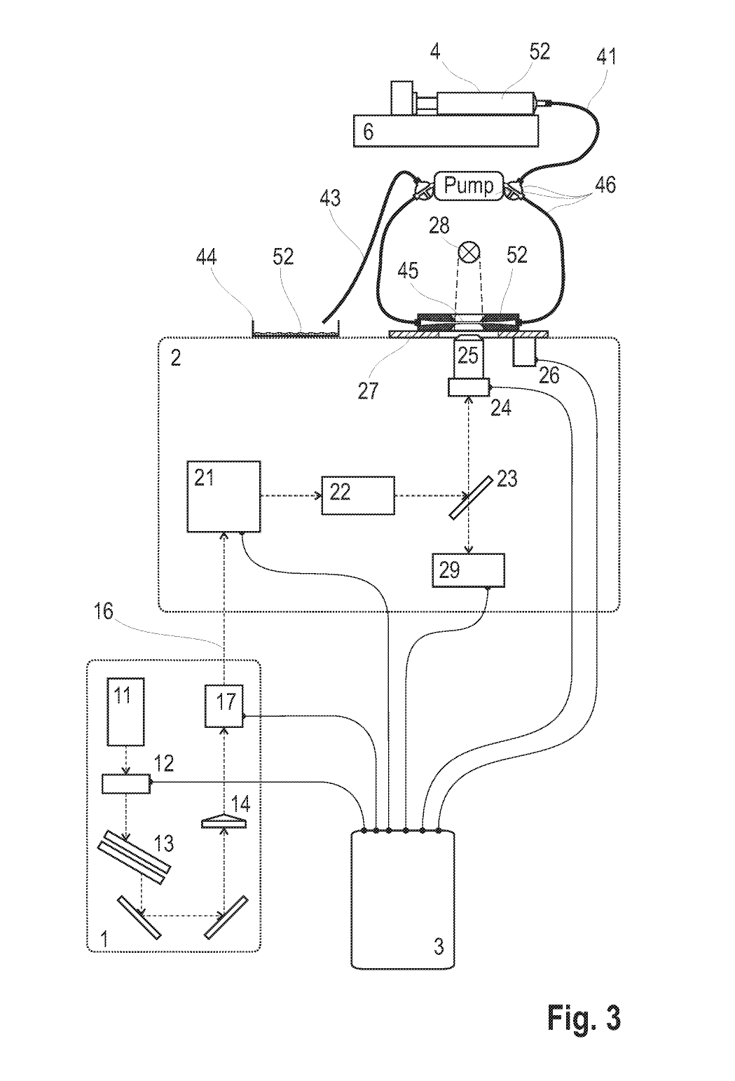

In a modified apparatus according to FIG. 3 in which only the construction of the flow cell 42 has been modified compared to FIG. 2, the cell suspension 52 flows through the micro-cannula 45 multiple times. To this end, a circulation system 46 of tubes is connected to the micro-cannula 45, a pump being installed therein so that the cell suspension 52 which has already been irradiated once flows through the micro-cannula 45 with two to three repetitions. A higher efficiency of the yield of cells which are optically reprogrammed multiple times can be achieved in this way. Since the cell membrane of a cell can also be perforated repeatedly without fatal damage to the cell, repeated consecutive perforation also does not pose an additional risk for the cells to be reprogrammed All of the rest of the steps and processes are carried out in the same manner as described with reference to FIG. 2.

### LIST OF REFERENCE NUMERALS

1 radiation source
11 femtosecond laser
12 shutter
13 dispersion compensator (chirped mirror)
14 Axikon element
15 periscope optics
16 laser beam
16′ laser beam
17 attenuation unit
2 laser scanning microscope
21 scanner
22 beam expansion
23 beamsplitter
24 focusing drive (z direction)
25 microscope objective
26 x-y table drive (x-y direction)
27 x-y table
28 illumination
29 video camera
3 control unit
4 supply receptacle
41 inlet line
42 flow cell
43 outlet line
44 cell chamber
45 micro-cannula
46 circulation system
5 sample
51 monolayer cells
52 cell suspension
6 metering device
7 incubator

What is claimed is:

1. A method for reprogramming living cells which makes use of transfecting a cocktail comprising at least two transcription factors and a microRNA into an interior of at least one living cell for converting it into an iPS cell or another type of cell, the method comprising:
supplying the cocktail without viral carriers in an aqueous environment of the at least one cell to be reprogrammed;
providing a radiation beam from a femtosecond laser with a pulse repetition frequency ranging between 50 MHz and 2 GHz and with a wavelength in a range from 700 to 1200 nm;
focusing the laser beam of the femtosecond laser by means of a laser scanning microscope with a numerical aperture of between 0.9 and 1.5 on a cell membrane of a selected cell in a sample with the at least one cell on a displaceable x-y table;
directing an attenuated nondestructive laser beam of the femtosecond laser for arranging and observing the at least one selected cell in the focus of the laser scanning microscope by means of a scanner; and
controlling a position of the at least one cell with respect to the focus, an exposure period and laser power for an optical treatment of the at least one selected cell such that the focused the laser beam depending on the pulse repetition frequency with an output between 7 mW and 100 mW generates a transient small-pore hole of a size up to 500 nm within the cell membrane of the to allow a diffusion of the virus-free cocktail for multiple reprogramming of the cell through an optical perforation of the membrane/through the optically perforated membrane.

2. The method according to claim 1, comprising carrying out irradiation for reprogramming by a femtosecond laser at a frequency between 75 and 85 MHz and with a center wavelength between 700 and 900 nm by means of the laser scanning microscope via a microscope objective with a numerical aperture between 1.1 and 1.3.

3. The method according to claim 2, comprising carrying out irradiation for reprogramming at an output of between 7 and 20 mW with pulse lengths between 5 fs and 20 fs and for a duration of between 0.2 and 1 second.

4. The method according to claim 2, comprising carrying out irradiation for converting at an output of between 50 and 100 mW with pulse lengths between 100 fs and 200 fs and for a duration of between 0.2 and 1 second.

5. The method according to claim 1, comprising providing before irradiation cells to be reprogrammed as monolayer cells on a glass substrate on an x-y table covered with the virus-free cocktail.

6. The method according to claim 1, comprising streaming cells to be reprogrammed as an aqueous cell suspension with the cocktail through a microfluidic flow cell with a micro-cannula.

7. The method according to claim 6, comprising carrying out irradiation for reprogramming with an output between 50 mW and 100 mW using a shaped laser beam in a Bessel beam mode with an elongated focus, wherein the Bessel beam mode forms the elongated focus over an diameter of the micro-cannula, wherein the scanner of the laser scanning microscope carries out a line scan orthogonal thereto so as to cover a cross-sectional area of the micro-cannula, and wherein the cell suspension flows through the micro-cannula with a flow rate of 135 to 145 nl/s.

8. The method according to claim 6, comprising circulating a flow through the flow cell so that the aqueous cell suspension of cells to be reprogrammed and the virus-free cocktail can stream through the micro-cannula repeatedly to increase a hit ratio of cells to be reprogrammed.

9. The method according to claim 1, comprising replacing the cocktail around the cells to be reprogrammed after a diffusion time of at least five seconds after irradiation by a plasmid-free medium, and incubating and storing the cells in an incubator for at least two days.

10. The method according to claim 9, comprising monitoring results of reprogramming during storing in the incubator by detecting GFP protein by means of a fluorescence microscope.

11. The method according to claim 1, comprising carrying out an optical multiple reprogramming of the at least one cell to an iPS cell by means of optical treatment.

12. The method according to claim 1, comprising carrying out a direct optical reprogramming by means of optical treatment of the at least one cell through conversion of one cell type into another cell type.

13. The method according to claim 1, comprising carrying out optical treatment in a tissue formed of a three-dimensional cell complex as perforation by boring channels with a diameter of up to 10 μm.

14. An apparatus for reprogramming living cells in which a cocktail made up of a required microRNA and at least two plasmids as transcription factors for the reprogramming is transfected into an interior of at least one cell to be reprogrammed into another type of cell, the apparatus comprising: a femtosecond laser for irradiation at a frequency between 75 and 85 MHz and with a center wavelength between 750 and 900 nm to generate a virus-free optical reprogramming through selective perforation of a cell membrane for transfecting the cocktail into an interior of the at least one cell to be reprogrammed; and a laser scanning microscope comprising the femtosecond laser and a microscope objective with a numerical aperture between 0.9 and 1.5, wherein the cells to be reprogrammed can be arranged in the microscope to be continuously selected for irradiation to achieve a perforation with at least one transient small-pore hole having a size up to 500 nm within a cell membrane of the at least one cell to be reprogrammed.

15. The apparatus according to claim 14, further comprising an x-y table for positioning monolayer cells on a glass substrate and for focusing a laser beam on the cell membrane of the monolayer cells.

16. The apparatus according to claim 15, wherein the femtosecond laser is configured as a laser with a frequency between 75 and 85 MHz, a pulse length between 10 fs and 20 fs and a center wavelength between 750 and 900 nm and which can be focused on the cell membrane by means of the microscope objective having a high numerical aperture in a range from 1.1 to 1.3 with a focus up to 500 nm.

17. The apparatus according to claim 14, wherein the x-y table comprises a microfluidic flow cell with a micro-cannula through which an aqueous cell suspension of the cocktail with the cells to be reprogrammed flows.

18. The apparatus according to claim 17, wherein the femtosecond laser is configured to emit a Bessel beam with an elongated focus, wherein a diameter of the micro-cannula is fully covered by an elongated focus of the Bessel beam that is moved in a line scan by means of a scanner of the laser scanning microscope such that the cells to be reprogrammed pass through the focus at a flow velocity generated by a flow rate between 135 and 145 nl/s with the micro-cannula diameter being 100 μm and are impinged while flowing through.

19. The apparatus according to claim 17, further comprising a cell chamber arranged downstream of the flow cell to capture the cell suspension of converted cells and to capture the cocktail for replacing the cocktail with a plasmid-free medium for storage in an incubator.
